(19) United States
(12) Patent Application Publication
(10) Pub. No.: US 2019/0046346 A1
(43) Pub. Date: Feb. 14, 2019

(54) Posture Correction Apparatus

(71) Applicant: Youreka Hub, LLC, San Antonio, TX (US)

(72) Inventor: Haley Waud, Chicago, IL (US)

(21) Appl. No.: 16/057,776

(22) Filed: Aug. 7, 2018

Related U.S. Application Data
(60) Provisional application No. 62/542,780, filed on Aug. 8, 2017.

(51) Int. Cl. A61F 5/02; A61H 3/04

(52) U.S. Cl. CPC A61F 5/028; A61H 2003/007; A61H 3/04

(57) ABSTRACT

A system and method for improving a user's posture when using a mobility assistance device, such as a wheeled walker, is disclosed. The system gently nudges a user to stand upright when a user is in a slumped or a bent posture. The user's posture may be approximated based on the distance between the user and the mobility assistance device, wherein, the greater the distance, the more likely it is that a user is in a slumped or a bent posture, and a shorter distance suggesting that the user is standing upright.

### CROSS-REFERENCE TO RELATED APPLICATIONS

This application claims priority to U.S. Provisional Patent Application Ser. No. 62/542,780, filed Aug. 8, 2017, entitled “POSTURE CORRECTING WALKER BELT” the entire content of which is incorporated herein by reference.

### BACKGROUND

### Field of the Art

This invention relates generally to the motion and physical health of the mammalian body, and more specifically to a posture correction apparatus for use with mobility assistance device devices for enabling users to correct their posture when using a mobility assistance device.

### Discussion of the State of the Art

When humans are unable to walk, or find it difficult to walk without assistance, they often turn to a mobility assistance device. These devices allow a user to place some of their bodyweight onto the device via their arms, thereby shifting the weight or the pressure from the user's legs to the user's arm, thus providing support, improving balance (by moving the center of gravity over the base of support), and decreasing fall risk. As a result of this weight redistribution, users typically experience a greater degree of motion in their legs, and therefore, are generally able to walk with the help of a mobility assistance device.

However, the additional range of motion often comes at the expense of poor posture. Because users who typically use mobility assistance devices have such limited strength and mobility in their lower extremities, they tend to compensate for this weakness by being overly reliant on their upper body strength and arms. For example, these users tend to push the walker forward, away from their body, by pushing the walker with their arms. But these users are typically much slower to bring their legs and feet closer to the mobility assistance device once it is extended out away from their body. As a result, users of mobility assistance devices tend to experience flexion of the trunk (including head and/or spine). For example, a user may lean his or her head/spine much further forward when using a mobility assistance device than a person who may not need to use a mobility assistance device. Specifically, these users' head and spine are not aligned to the rest of their bodies.

The damage associated with poor posture is well documented in humans. This damage may be exacerbated in individuals with loss of function in their lower limbs. Generally, poor posture places undue stress on a variety of different parts of a human body and causes, for example, neck pain, back pain, knee pain, and the like. As a result, users of mobility assistance devices may experience long term damage resulting from their use of a mobility assistance device, and poor posture that is typically associated with the use.

Some have tried to alleviate this problem by creating mobility assistance devices wherein users sit in a harness that is connected to the mobility assistance device. But these devices, nonetheless, require assistance from a caregiver, who generally has to help the user move around in the wheeled walker. Moreover, these devices are notoriously difficult to get in-and-out of without additional assistance from a caregiver and the like. As a result, these solutions tend not to improve the mobility of users who wish to move around without assistance from a caregiver. Finally, these devices do very little to reduce the expense associated with paying caregivers who are generally still needed despite efforts documented in prior art.

What is needed, is a means to assists users correct and improve their posture when using a mobility assistance device, and, at the same time, reduce reliance on assistance from caregivers who may be expensive or otherwise unavailable when a user wishes to move about.

### SUMMARY

Accordingly, the inventor has conceived and reduced to practice, an apparatus and a method for ensuring that humans who use a mobility assistance device maintain good posture while using the device. Good posture may be defined in a variety of ways, as would be readily apparent to a person of ordinary skill in the art, but the inventive apparatus specifically helps users improve their posture by helping them keep their upper body and head substantially on top of, and aligned with their torso and lower body. In other words, the inventive apparatus ensures that a user's trunk does not experience flexion (e.g. head and upper body do not extend too far forward), and put undue pressure on the spine and cause it to bend in an unnatural direction, including, but not limited to a forward direction.

According to one aspect, the apparatus comprises a belt that may attach to a mobility assistance device and a user. In one embodiment, the apparatus sets the maximum distance that a user may be able to travel away from the apparatus. For example, in one embodiment, the apparatus may prevent a user from extending the walker too far forward such that the user's spine comes out of alignment from the rest of the user's body. Or, in other words, it prevents users from extending his or her torso too far forward from the user's legs.

According to one aspect, the method of using the inventive apparatus comprises the steps of attaching the apparatus to the user, attaching the apparatus to the mobility assistance device, and adjusting the length of the straps that connect the user to the mobility assistance device. A proper fitting, or an adjustment of the maximum distance permissible by the apparatus, ensures that the user's spine does not come out of alignment with the rest of the user's body.

According to one aspect, the apparatus and the method disclosed herein do not limit a user's mobility, and do not otherwise interfere with a user's ability to use a mobility assistance device. For example, the inventive apparatus does not limit the length of the strides that a user must make while using the apparatus. Moreover, the inventive apparatus does not force users to support more weight through their arms than would be otherwise required in using a mobility assistance device without the inventive apparatus. As such, the disclosed apparatus is also easy to use. Specifically, the apparatus is designed so that a user, whose mobility is impaired or limited, may be able to use the apparatus, and take it on-and-off, without requiring significant dexterity or flexibility. Indeed, a user does not have to bend, twist, or turn significantly in order to take the device on-or-off, or to use it. Similarly, the inventive apparatus is designed so that a user can take it on-and-off without assistance from another person. As such, the inventive apparatus improves the mobility and the quality of life for mobility impaired users without adding significant costs or the need to employ assistance.

In one embodiment, the inventive apparatus may be a wheeled walker. For example, the inventive apparatus may be manufactured and sold as a singular item. In another aspect of the invention, the inventive apparatus may be removable from a mobility assistance device, and a may be configured to attach to a large variety of different mobility assistance device to ensure universal compatibility and fit, regardless of their shape, size, origin of manufacture, etc.

### BRIEF DESCRIPTION OF THE DRAWING FIGURES

The accompanying drawings illustrate several embodiments and, together with the description, explain the principles of the invention according to the embodiments. It will be appreciated by one skilled in the art that the arrangements illustrated in the drawings are merely exemplary and are not to be considered as limiting of the scope of the invention or the claims herein in any way.

FIG. 1 illustrates, in accordance to one embodiment of the invention, a posture correction apparatus.

FIG. 2a illustrates, from a side view, a user using a posture correction apparatus that is attached to a wheeled walker, in accordance with one embodiment of the invention.

FIG. 2b illustrates, from a side perspective view, a user using a posture correction apparatus that is attached to a wheeled walker, in accordance with one embodiment of the invention.

FIG. 3 illustrates a posture correction apparatus in accordance with one embodiment of the invention.

FIG. 4a illustrates a portion of the posture correction apparatus and a walker, in accordance with one embodiment of the invention.

FIG. 4b illustrates a portion of the posture correction apparatus and a walker, in accordance with one embodiment of the invention.

FIG. 5 is a flow diagram illustrating an exemplary method for using the posture correction apparatus, in accordance with one embodiment of the invention.

### DETAILED DESCRIPTION

The inventor has conceived, and reduced to practice, a system and method for correcting a user's posture while he or she is using an assistive apparatus, such as a walker.

One or more different embodiments may be described in the present application. Further, for one or more of the embodiments described herein, numerous alternative arrangements may be described; it should be appreciated that these are presented for illustrative purposes only and are not limiting of the embodiments contained herein or the claims presented herein in any way. One or more of the arrangements may be widely applicable to numerous embodiments, as may be clear from the disclosure. In general, arrangements are described in sufficient detail to enable those skilled in the art to practice one or more of the embodiments, and it should be appreciated that other arrangements may be utilized and that structural, logical, software, electrical and other changes may be made without departing from the scope of the embodiments. Particular features of one or more of the embodiments described herein may be described with reference to one or more particular embodiments or figures that form a part of the present disclosure, and in which are shown, by way of illustration, specific arrangements of one or more of the aspects. It should be appreciated, however, that such features are not limited to usage in the one or more particular embodiments or figures with reference to which they are described. The present disclosure is neither a literal description of all arrangements of one or more of the embodiments nor a listing of features of one or more of the embodiments that must be present in all arrangements.

Headings of sections provided in this patent application and the title of this patent application are for convenience only and are not to be taken as limiting the disclosure in any way.

Devices and parts that are connected to each other need not be in continuous connection with each other, unless expressly specified otherwise. In addition, devices and parts that are connected with each other may be connected directly or indirectly through one or more connection means or intermediaries.

A description of an aspect with several components in connection with each other does not imply that all such components are required. To the contrary, a variety of optional components may be described to illustrate a wide variety of possible embodiments and in order to more fully illustrate one or more embodiments. Similarly, although process steps, method steps, or the like may be described in a sequential order, such processes and methods may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the embodiments, and does not imply that the illustrated process is preferred. Also, steps are generally described once per aspect, but this does not mean they must occur once, or that they may only occur once each time a process, or method is carried out or executed. Some steps may be omitted in some embodiments or some occurrences, or some steps may be executed more than once in a given aspect or occurrence.

When a single device or article is described herein, it will be readily apparent that more than one device or article may be used in place of a single device or article. Similarly, where more than one device or article is described herein, it will be readily apparent that a single device or article may be used in place of the more than one device or article.

The functionality or the features of a device may be alternatively embodied by one or more other devices that are not explicitly described as having such functionality or features. Thus, other embodiments need not include the device itself.

Techniques and mechanisms described or referenced herein will sometimes be described in singular form for clarity. However, it should be appreciated that particular embodiments may include multiple iterations of a technique or multiple instantiations of a mechanism unless noted otherwise. Alternate implementations are included within the scope of various embodiments in which, for example, functions may be executed out of order from that shown or discussed, including substantially concurrently or in reverse order, depending on the functionality involved, as would be understood by those having ordinary skill in the art.

### Overview

The inventive apparatus described herein helps users maintain proper posture—and improve their posture as necessary—when using a mobility assistance device, including, for example, but not limited to, a wheeled-walker, non-wheeled walker, a cane, a gait training device, a body weight support system, or the like. Generally, any reference to a mobility assistance device herein refers to each of the devices listed herein as well as others known to a person of ordinary skill. Any reference in this application to a wheeled-walker, or any of the other devices, should be interpreted to refer to all of the other devices listed herein as well as others known to a person of ordinary skill in the art.

In one embodiment of the invention, a portion of the inventive apparatus attaches to a user's lower torso, and another portion of the apparatus attaches to a mobility assistance device. The two portions may be connected by a connection portion, such that, the apparatus gently nudges the user forward and to stand upright whenever a maximum distance between the user and the mobility assistance device is reached.

Generally, an approximation of the user's posture, and the distance between the user and the mobility assistance device, is determined by the connection portion. For example, the greater the distance between the user and the mobility assistance device, the more likely it is that a user is in a slumped or a bent posture. On the other hand, the shorter the distance, the more likely it is that the user is in an upright posture. The appropriate distance between a user and a mobility assistance device may vary from user to user depending on the user's height, weight, gait, length of limbs, etc. In one embodiment, the appropriate distance between the user and the mobility assistance device may be set at a fitting, which may be performed by a healthcare provider, caregiver, or the users themselves. Generally, the connection portion may be adjustable to increase or decrease the distance between the user and the mobility assistance device.

### Posture Correction Apparatus

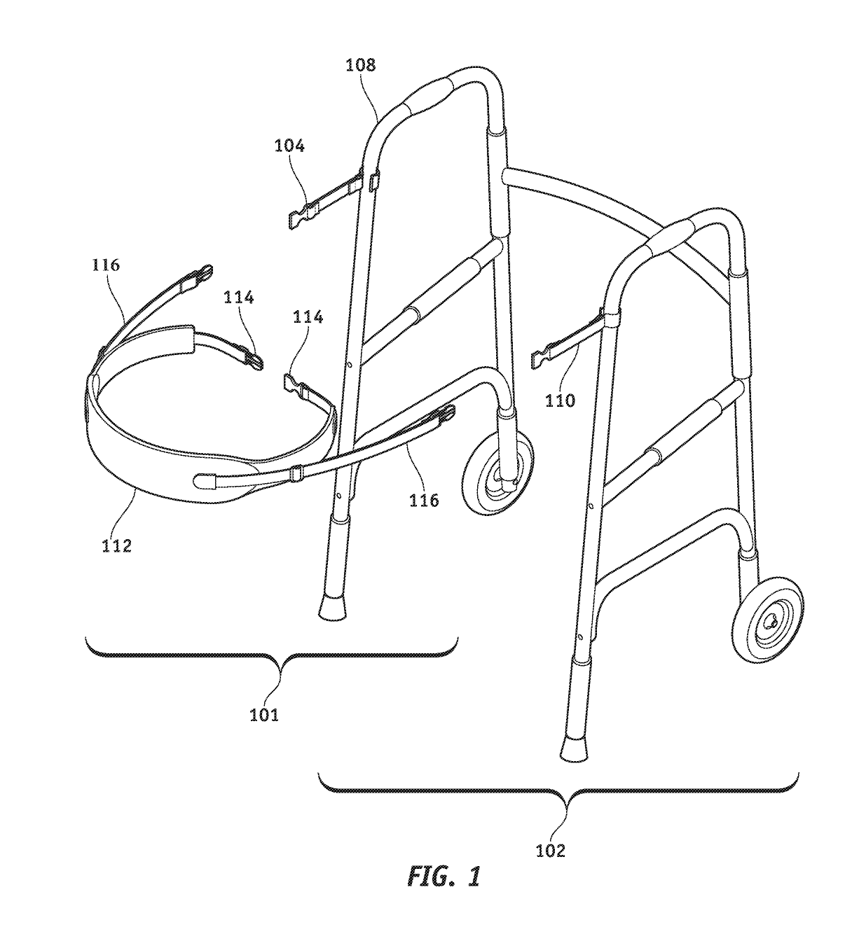

FIG. 1 illustrates one exemplary embodiment of the posture correction apparatus 101 and an exemplary mobility assistance device 102. The posture correction apparatus 101 comprises a first attachment portion 116, a second attachment portion 114, a back-support pad 112, and a second attachment portion 114. In this particular figure, the mobility assistance device 102 is a wheeled walker; it comprises a traction portion (i.e. two legs that prevent the walker 102 from moving in a lateral direction), wheels that help the walker 102 move in a lateral direction, handlebars that permit a user to raise the traction portion off the ground and push the walker 102 in a lateral direction, and some optional bracing for improving the structural integrity of the walker 102.

Generally, the posture correction apparatus 101 connects to a mobility assistance device 102 via the first attachment portion 116, and connects to a user via the second attachment portion 114. The length of the second attachment portion 114 generally sets the maximum distance that a user may be away from a mobility assistance device while connected the posture correction apparatus 101 via the second attachment portion 114, and the mobility assistance device via the first attachment portion 116.

In one embodiment, the posture correction apparatus 101 connects to a mobility assistance device 102 via device straps 104. The device straps 104 comprise a first coupling attachment 108 and a second coupling attachment 110. The first coupling attachment 108 connects to the mobility assistance device 102, and the second coupling attachment 110 connects to the posture correction apparatus 101.

In one alternate embodiment, the device strap 104 and the second attachment portion 114 are comprised of one component, not two separate components. In this embodiment, the posture correction apparatus 101 includes two straps, one that attaches to a user, and one that attaches to a mobility assistance device.

In yet another alternative embodiment, the posture correction apparatus 101 may be integrally formed with the mobility assistance device 102. In one such an instance the device strap 104 may not be removable from the mobility assistance device 102. In another instance, alternatively, but not necessarily, at least one second coupling attachment 110 may not be detachable from the posture correction apparatus 101. In such an instance, a user may be able to attach or detach from the mobility assistance device by using the second attachment mechanism 114 and at least one detachable second coupling mechanism 110.

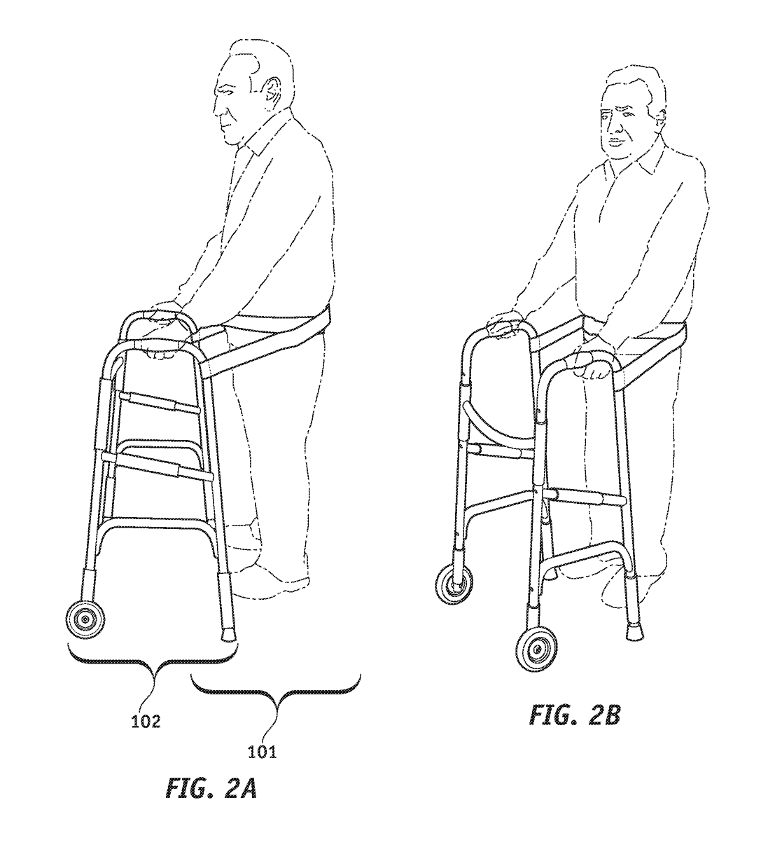

FIGS. 2a and 2b illustrate a user who is using the inventive posture correction apparatus that is attached to a wheeled walker via a device strap 104, and to the user via a second attachment portion 114. As can be seen in the figures, the posture correction apparatus 101, via the second attachment portion 114, sets a maximum distance that a mobility assistance device 102 may travel away from the user when the posture correction device 101 is attached to a user and the mobility assistance device 102. As shown in FIGS. 2a and 2b, the second attachment portion 114 securely attaches to a user's lower torso, and the first attachment portion 116 attaches to the mobility assistance device 102. When attached in this manner, the mobility assistance device 101 gently nudges the user to move closer to the mobility assistance device 102, thereby causing the user to stand upright, with a better posture, which causes the user's back, spine, and head to be aligned. Generally, the user's posture, including the user's back, spine, and head, come out of alignment when the mobility assistance device 102 moves too far away from the user, thereby causing the user has to bend forward to support his or her weight on the mobility assistance device 102 and thereby increasing the risk of injury related to poor posture, including the risk of falling, or risk typically associated with chronic or repetitive stress type of injury.

### First Attachment Portion

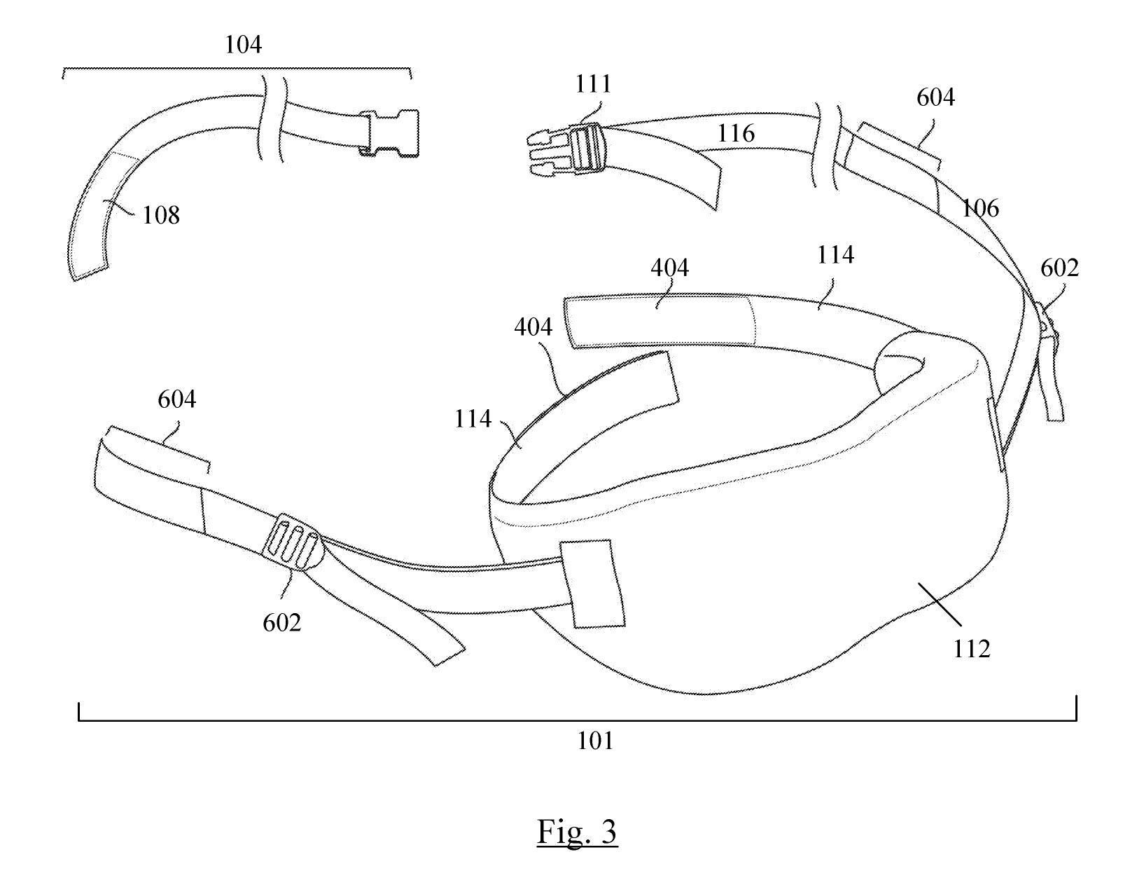

FIG. 3 illustrates an exemplary posture correction apparatus 101, including a first attachment portion 116. In one embodiment, the first attachment portion 104 connects the posture correction apparatus 101 with a mobility assistance device 102.

FIG. 3 illustrates the first attachment portion 116 as a strap that connects to a posture correction apparatus 101 on one end, and a mobility assistance device 102 on the other end. The straps are exemplary and are for illustration purposes only; they are intended to be non-limiting, and are illustrated herein for convenience purposes only. Any mechanism for connecting the apparatus 101 to a mobility assistance device 102 may be used without departing from the scope of the invention. Other examples for connecting the inventive apparatus 101 to a mobility assistance device 102 include, but are not limited to, wires, belts, magnets, magnetic fields, gait belts, webbing, etc.

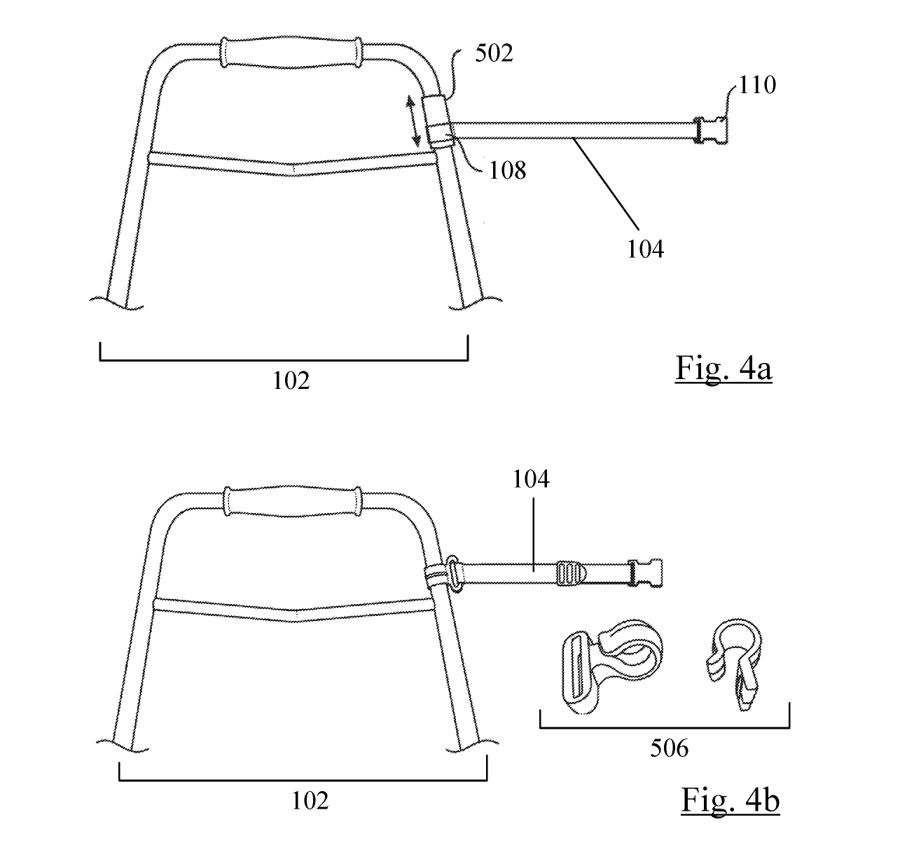

In one embodiment, the first attachment portion 116 attaches directly to a mobility assistance device 102. For example, in one embodiment, the first attachment portion's hoop and loop closure attaches around a portion of the mobility assistance device. In another alternative embodiment, as illustrated in FIGS. 4a and 4b, and as described in greater detail below, the first attachment portion 116 connects to a device strap 104 that connects to the mobility assistance device 102. In such an embodiment, the first attachment portion 116 may attach to a device strap 104 via an attachment mechanism that may be known to a person of ordinary skill in the art, including, but not limited to side-release buckles, D-rings, hook and loop closures, magnets, etc.

In one embodiment of the invention, the first attachment portion 116 is adjustable. Accordingly, it may be possible to increase or decrease the length of the attachment portion 116 as appropriate or as desired by a user. Any mechanism for adjusting the length of the first attachment portion 116 may be used without departing from the scope of the invention. Exemplary methods for adjusting the length of the first attachment portion 116 include, but are not limited to, D-rings, ladder locks, stretchable webbing or material, etc. As noted above, the first attachment portion 116 helps users maintain proper posture while using a mobility assistance device by setting a maximum distance between the walker and the user to ensure that the walker does not get too far away from the user. In other words, the length of the first attachment portion 116 ensures that the walker does not move away from the user beyond the length of the first attachment portion 116. As such, the length of the first attachment portion 116 may be adjusted to ensure that inventive apparatus 101 helps users correct their posture by ensuring that the users do not travel too far from the walker, and do not put excessive weight on it through their forearms and wrists. In an alternative embodiment wherein device straps 104 are used to connect the apparatus 101 to the mobility assistance device 102, the first attachment portion 116 and/or one or more device straps 104 (which is described in greater detail below) may be adjustable.

In one instance, the length of the first attachment portion 116 may be set when a user is fitted for a walker, or may be adjusted by a user, a healthcare provider, or a caregiver. The ideal distance between a user may be set according to the angle of elbow flexion of the user while using the wheeled walker. The appropriate distance, and therefore the appropriate length of the first attachment portion 116, may be set by the user, healthcare provider, or a caregiver during a fitting.

In one embodiment, the first attachment portion is connectable and disconnect-able to a mobility assistance device 101 as described in greater detail below.

### First Attachment Mechanism

FIGS. 4a and 4b illustrate two exemplary methods for connecting the apparatus 101 to a mobility assistance device 102. Although the figures show a device strap 104 being attached to a mobility assistance device via a first attachment mechanism 108 (as illustrated in FIG. 4a) and 506 (as illustrated in FIG. 4b), the same attachment mechanisms 108, 506 may be used to connect the first attachment portion 116 to a mobility assistance device 102 in embodiment wherein a device strap 104 is not used.

FIG. 4a illustrates the use of a hook and loop closure system for attaching the device strap 104 to a mobility assistance device 102. In this embodiment, a first hook and loop closure strip 502 is attached to the mobility assistance device 102 at one or more locations on the device 102. The hook and loop closure strip 502 attaches to a second, complementary hook and loop closure piece 108 on the device strap 104. Thus, when a user wants to connect the device strap 104 to the mobility assistance device 102, the user may simply attach element 108 to element 502. In one embodiment, a first hook an loop closure strip 502 may not be used. A hook and loop closure strip 108 comprising two complimentary pieces may be used to simply tie or attach the first attachment portion 116 or the device strap 104 around a mobility assistance device. In one embodiment, the device strap 104 or the first attachment mechanism 116 may include a pull tab that enables a user to easily disconnect the apparatus from a mobility assistance device when the apparatus is attached to the mobility assistance device with a hook and loop closure system.

FIG. 4b illustrates another method for connecting the first attachment portion 116 or a device strap 104 to a mobility assistance device 102. In one embodiment, the first attachment portion 116 and/or the device strap 104 includes clips 506 that attach and/or hook around a portion of the mobility assistance device. The clips 506 may be plastic or may be made from any other material that may be chosen by a person of ordinary skill in the art as appropriate.

The invention described herein is not limited to the first attachment mechanisms described here, and illustrated in FIGS. 4a and 4b. Any attachment mechanism known to a person of ordinary skill may be used to without departing from the scope of the invention.

In one embodiment, the first attachment portion 116 or the device strap 104 may be formed integrally with posture correction apparatus 101. In another embodiments, as illustrated in FIGS. 4a and 4b, the first attachment portion 116 may connect to the device straps 104 via a device connection mechanism 110. FIGS. 4a and 4b specifically illustrate side-release buckles as an exemplary mechanism for connecting the device straps 104 to the first attachment portion 116. But other mechanism, which may be apparent to a person of ordinary skill in the art, may be used without departing from the scope of the invention, including, but not limited to buckles, D-rings, magnets, fasteners, clips, etc.

In one embodiment of the invention, the attachment mechanism for attaching the first attachment mechanism 116 and/or the device strap 104 to the mobility assistance device 102 may unplug or disconnect automatically when sufficient force is applied. Any mechanism known to a person of ordinary skill in the art may be used. This mechanism ensures that the inventive apparatus does not become a hazard to the user. For example, the auto-disconnect feature prevents injury if the user becomes entangled in the device, or if the device gets away from the user or the user begins to fall.

### Second Attachment Portion

Referring again to FIG. 3, it illustrates the various components of the inventive apparatus in greater detail, including a second attachment portion 114. The second attachment portion 114 attaches around a user's lower torso, including the user's hip, stomach, or abdominal area, as illustrated in FIGS. 2a and 2b. Generally, the second attachment portion 114, in conjunction with the device strap 104, keeps the mobility assistance device from moving too far away from the user by forming a closed loop between the user and the mobility assistance device. In one embodiment, the second attachment portion 114 may include the use of gait straps that wrap around a user's torso.

FIG. 3 illustrates the second attachment portion 114 as a strap that connects around a user. The straps are exemplary and are for illustration purposes only; they are intended to be non-limiting, and are illustrated herein for convenience purposes only. Any mechanism for connecting the apparatus 101 to a user may be used without departing from the scope of the invention. Other examples for connecting the inventive apparatus 101 to a mobility assistance device 102 includes, but are not limited to, wires, belts, magnets, magnetic fields, gait belts, etc. For example, in one embodiment, the second attachment portion 114 includes the use of a gait belt.

In one embodiment, the second attachment portion 114 does not wrap around a user's torso. In such an instance, the second attachment portion 114 is comprised of one or more pieces that attach to a user's hip on either side. This configuration is helpful, for example, if the user has one side that is weaker than the other. By selectively attaching the posture correction apparatus 101 to the weaker side, the user may be able to train the weaker side, and/or ensure that the mobility assistance device does not travel too far away from the user's weaker side. This embodiment may also be helpful if the user cannot enclose his or her torso with a second attachment portion 114.

In one embodiment, the second attachment portion 114 is adjustable. For example, the second attachment portion 114 may be lengthened or shortened to fit a variety of different users with a variety of different sizes. Any mechanism known to a person of ordinary skill in the art for adjusting the length of the attachment portion 114 may be used, including, without limitation, D-rings, ladder locks, stretchable webbing or materials, etc., without departing from the scope of the invention.

In one exemplary embodiment, the second attachment portion 114 may include a cushioned element 112, as illustrated in FIG. 3. The cushioned element provides additional comfort and support the user by ensuring that the second attachment portion 114 does not feel sharp or abrasive against the user's body.

In one embodiment, the second attachment portion 114 is connectable and disconnect-able to a mobility assistance device 101 as described in greater detail below.

### Second Attachment Mechanism

FIG. 3 also illustrates a second attachment mechanism 404 for attaching the second attachment portion 114 to a user. Any attachment mechanism that is known to a person of ordinary skill in the art may be used, including, without limitation, a hook and loop closure system, a belt system, D-rings, side release buckles, magnets, etc.

In one embodiment, the attachment mechanism 114 is adjustable, and/or is easy to take on and off. The adjustable nature of the attachment mechanism 114 ensures that it easily fits around users of a variety of different shapes and sizes. The easy on-off mechanism permits a user to use the device more easily.

### Connection Portion

FIG. 3 also illustrate a connection portion 106 that connects the first attachment portion 116 to the second attachment portion 114. In one embodiment the connection portion 106 may connect to the device strap 104 on one end, and a second attachment portion 114 on the other end. In other embodiments, the connection portion 106 may be integrally formed with either or both the first attachment portion 116 and/or the second attachment portion 114.

FIG. 3 illustrates the connection portion 106 as a strap. However, the straps are exemplary and are for illustration purposes only; they are intended to be non-limiting, and are illustrated herein for convenience purposes only. Any mechanism or material for connecting the first attachment portion 116 and the second attachment portion 114 may be used without departing from the scope of the invention. Other examples of mechanisms or materials used in the connection portion 105 include, but are not limited to, wires, belts, magnets, magnetic fields, gait belts, etc.

In one embodiment, the connection portion 106 may be comprised of a stretchable material 604. For example, a stretchable material 604 may be used along the length of the connection portion 106. This feature makes the posture correction apparatus 101 easier to use. If a user makes an error and if a mobility assistance device travels too far from the user's body, then the give on the connection portion 106 will prevent the user from being jerked towards the mobility assistance device. In one embodiment, the stretch material 604 may also, or in the alternative, be incorporated in the device strap 104.

In one embodiment, the attachment mechanism 114 automatically disconnects when sufficient pressure is applied to it. For example, if a user falls or somehow gets entangled in the device, the automatic disconnect feature permits the user to remove the second attachment portion 114 quickly and easily.

In one embodiment, the connection portion 106 is adjustable. As illustrated in FIG. 3, in one exemplary embodiment, the connection portion 106 may include a mechanism 602 for adjusting the length of the connection portion 106. The adjustment mechanism 602 enables a user, or a physical therapist, to lengthen or shorten the distance between the user and the mobility assistance device. Any known method for adjusting the length of the connection portion 106 may be used, including, but not limited to D-rings, ladder locks, stretchable webbing or material, etc. The adjustment mechanism 602 makes the inventive apparatus customizable so that it can be used appropriately and safely by a variety of users, including those with different gaits and stride lengths, and other physiological factors.

A variety of different systems known to a person of ordinary skill in the art may be used as an adjustment mechanism 106. For example, a D-ring may be used as an adjustment mechanism 602 for adjusting the length of a portion of the second attachment portion 114. Other mechanisms known to a person of ordinary skill in the art may also be employed, including, but not limited to hook and loop closure systems, slider buckles, belts, etc.

In an alternate embodiment, the adjustment mechanism 602 may be disposed in the device strap 104 and/or the first attachment portion 116 and/or the connection portion 106. As noted above, in embodiments wherein the device strap 104 and/or the first attachment portion 116 and/or the connection portion 106 and/or the second attachment portion 114 are one unit, the adjustment mechanism may be disposed in the combined unit.

In one embodiment, the connection portion 106 is connectable and disconnect-able to one or more of the first attachment portion 116 and the second attachment portion 114 as described in greater detail below.

### Connection Mechanism

The connection portion 106 may connect to the first attachment portion 116 and/or the second attachment portion 114 via a variety of different connection mechanisms 111, including, without limitation, side clasp buckles, as illustrated by element 111, hook and loop closure system, magnets, etc. The connection mechanism 111 must be strong enough to ensure that the user remains connected to the mobility assistance device during normal use, but also must be easy to attach and detach to ensure that the user can quickly and easily remove the inventive apparatus when it is not needed. In one embodiment, the connection mechanism 111 automatically detaches when an unusual amount of force is applied to it. For example, it the user falls or somehow becomes entangled in the device, then the connection mechanism 111 disconnects so that the user can safely extricate himself or herself from the inventive apparatus.

### Method for Using a Posture Correction Apparatus

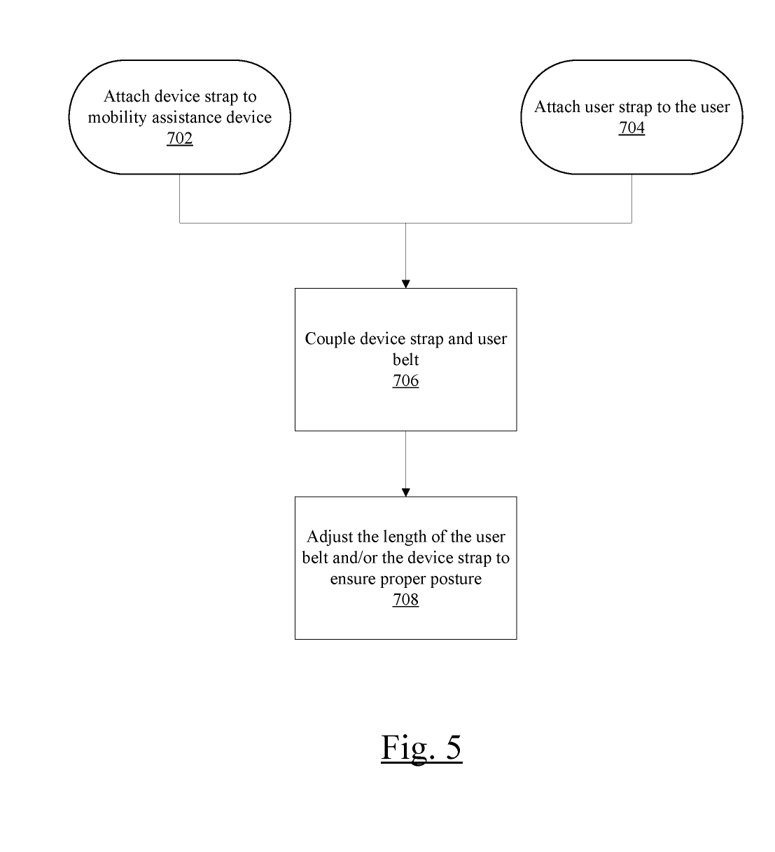

A process for using the posture correction apparatus is illustrated in FIG. 5. It includes the steps of attaching a first attachment portion to a mobility assistance device 503, attaching a second attachment portion to a user 505, connecting the first attachment portion with the second attachment portion 507, and adjusting the adjustment portion 509 to ensure that the user has proper posture when using the posture correction apparatus. These steps are merely illustrative. They may be performed in a variety of different orders without departing from the scope of the invention.

Step 503 is comprised of attaching a first attachment portion to a mobility assistance device. In one embodiment, the first attachment portion may be attached via a first attachment mechanism, which may be provided with the inventive apparatus. For example, if, in one embodiment, the attachment mechanism includes the use of hook and loop closure, then step 503 comprises attaching one part of the hook and loop closure strip to the mobility assistance device, and coupling it with a complimentary hook and loop closure strip on the first attachment portion. If the attachment mechanism is comprised of plastic clips, then the step 503 of attaching the first attachment portion to a mobility assistance device comprises attaching the one or more plastic clips to the mobility assistance device. This coupling ensures that the posture correction apparatus is connected to the mobility assistance device.

Step 505 is comprised of attaching a second attachment portion to a user. In one embodiment, this step comprises using an attachment mechanism that is provided with the inventive apparatus. For example, if, in one embodiment, the attachment mechanism includes the use of hook and loop closure strips, then step 505 may be performed by coupling the two complementary strips of hook and loop closure strips around a user's torso, hips, or abdominal area. If the attachment mechanism comprises side clasping buckles, then the step 505 may be performed by simply coupling the two side clasping buckles. Thus, step 505 of attaching a user belt to a user may vary based on the attachment mechanism that is provided with the inventive apparatus. Step 505 ensures that the posture correction apparatus is connected to the user.

Step 507 is comprised of attaching the first attachment portion with a second attachment portion. This step comprises using the attachment mechanism that is provided with the inventive apparatus. For example, if, in one embodiment, the attachment mechanism includes the use of hook and loop closure strip, then step 507 may be performed by coupling the complementary hook and loop closure strips on each the first attachment portion, and the second attachment portion. The specific methodology for carrying out step 507 may vary based on the attachment mechanism that is provided with the inventive apparatus. Step 507 ensures that the posture correction apparatus connects the user to a mobility assistance device.

In an embodiment, wherein the device strap and the user belt are one component, as opposed to two separate components, the user need not perform step 507 because the two components are pre-attached.

Step 509 is comprised of adjusting the length of the device strap and/or the user belt. This step ensures that the user has proper posture when he or she is using the mobility assistance device. As described above, a user may have poor posture when using a mobility assistance device if the mobility assistance device is too tall or too short for the user, or if the device is too close or too far from the user. The adjustment step 509 sets a maximum distance between the user and the mobility assistance device. A proper adjustment would ensure that the user is not leaning too far forward or placing an undue amount of weight on his or her palms and/or wrists when using the mobility assistance device. The amount of adjustment needed for each user may vary. Moreover, the amount of adjustment that is applied to each, device strap or user belt may vary depending on the user's or the adjustor's preferences. In some embodiment, it may not be possible to adjust one of the first attachment portion or the second attachment portion. In such an embodiment, the adjustment may be applied to the portion of the posture correction apparatus that is adjustable.

The skilled person will be aware of a range of possible modifications of the various embodiments described above. Accordingly, the present invention is defined by the claims and their equivalents.

Upon reading this disclosure, those of skill in the art will appreciate still additional alternative structural and functional designs through the disclosed principles herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims. Accordingly, it should be understood that the invention is not limited to the above-described embodiments, but may be modified into various forms on the basis of the spirit of the invention. Additionally, the modifications are included in the scope of the invention.

I claim:

1. An apparatus for improving the posture of a user who is using a mobility assistance device, comprising:
a first attachment mechanism for attaching the apparatus to a mobility assistance device;
a second attachment mechanism for attaching the apparatus to a user; and
a connection portion connecting the first attachment mechanism to the second attachment mechanism, wherein the length of the connection portion sets the maximum distance between the mobility assistance device and the user when the apparatus is attached to both the mobility assistance device and the user.

2. The apparatus of claim 1, wherein the length of the connection portion is adjustable.

3. The apparatus of claim 1, wherein the connection portion includes a connection mechanism that is connectable and disconnect-able.

4. The apparatus of claim 3, wherein the connection mechanism automatically disconnects when sufficient pressure is applied between the first attachment portion and the second attachment portion.

5. The apparatus of claim 1, wherein the connection portion is stretchable, wherein the connection portion may stretch upon application of sufficient pressure, thereby gently urging a user to correct his or her posture when the distance between the user and the mobility assistance device is greater than permissible by the unstretched connection portion.

6. The apparatus of claim 1, wherein the first attachment mechanism is removably attachable to a mobility assistance device.

7. The apparatus of claim 6, wherein the first attachment mechanism comprises hooks that securely connect to a mobility assistance device by hooking around a portion of the mobility assistance device.

8. The apparatus of claim 6, wherein the first attachment mechanism comprises a hook and loop fastener that securely fastens around a portion of the mobility assistance device.

9. The apparatus of claim 1, wherein the second attachment mechanism attaches the apparatus to a user's lower torso.

10. The apparatus of claim 1, wherein the second attachment mechanism is removably attachable to a user.

11. The apparatus of claim 10, wherein the second attachment mechanism is comprised of side release buckles, which enable a user to attach or disconnect the second attachment portion.

12. The apparatus of claim 10, wherein the second attachment mechanism is comprised of complimentary hook and loop fasteners that securely attach around a user's lower torso.

13. The apparatus of claim 1, wherein the second attachment mechanism is adjustable, wherein the attachment portion may be used by users of a variety of different sizes.

14. The apparatus of claim 1, wherein the apparatus further comprises a cushion portion.

15. The apparatus of claim 1, wherein the mobility assistance device comprises a walker.

16. The apparatus of claim 1, wherein the mobility assistance device comprises a wheeled walker.

17. A wheeled walker comprising:
a posture correction apparatus that, when worn by a user, urges the user to stand upright when the user is in a slumped posture as determined by the distance between the user and the wheeled walker;
a first attachment mechanism that connects the wheeled walker to the posture correction apparatus;
a second attachment mechanism for attaching the posture correction apparatus to a user; and
an adjustment mechanism for setting the maximum allowable distance between a user and the wheeled walker when the posture correction apparatus is attached to the wheeled walker and also attached to the user.

18. The apparatus of claim 17, wherein the adjustment mechanism is adjustable.

19. The apparatus of claim 17, wherein the second attachment mechanism is connectable and disconnect-able.

20. A method of using a posture correction apparatus for improving the posture of a user who is using a mobility assistance device, the method comprising:
attaching a first portion the posture correction apparatus to the mobility assistance device;
attaching a second portion the posture correction apparatus to the user;
connecting, with a connection portion, the first portion and the second portion of the posture correction apparatus;
adjusting the length of the connection portion to set the maximum distance between the mobility assistance device and the user, wherein the posture correction apparatus gently nudges the user to move closer to the posture correction apparatus, and thereby stand upright when the maximum distance between the user and the mobility assistance device is reached.
